(19) United States
(12) Patent Application Publication
(10) Pub. No.: US 2026/0183319 A1
(43) Pub. Date: Jul. 2, 2026

(54) PHARMACEUTICAL COMPOSITION COMPRISING NEUROKININ-1 ANTAGONIST PRODRUG COMPOUND

(71) Applicant: JIANGSU HENGRUI PHARMACEUTICALS CO., LTD., Lianyungang, Jiangsu (CN)

(72) Inventors: Mingzhu JU, Lianyungang (CN); Xiaoyan WANG, Lianyungang (CN); Jie WANG, Lianyungang (CN); Xiaoli CAO, Lianyungang (CN)

(21) Appl. No.: 18/728,162

(22) PCT Filed: Jan. 12, 2023
(86) PCT No.: PCT/CN2023/071901

§ 371 Date: Jul. 11, 2024

(30) Foreign Application Priority Data
Jan. 12, 2022 (CN) 202210032508.4

(51) Int. Cl. A61K 31/675; A61K 9/08; A61K 9/19; A61K 31/4178; A61K 31/4184; A61K 31/439; A61K 31/46; A61K 31/473; A61K 47/18; A61K 47/22; A61K 47/26

(52) U.S. Cl. CPC A61K 31/675; A61K 9/08; A61K 9/19; A61K 31/4178; A61K 31/4184; A61K 31/439; A61K 31/46; A61K 31/473; A61K 47/183; A61K 47/22; A61K 47/26

(57) ABSTRACT

A pharmaceutical composition comprising a neurokinin-1 antagonist prodrug compound. A pharmaceutical composition comprising a compound of formula I and a stabilizer, and a preparation method therefor.

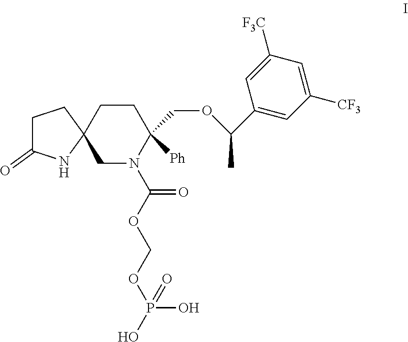 [SMILES: C[C@@H](OC[C@@]1(c2ccccc2)CC[C@]2(CCC(=O)N2)CN1C(=O)OCOP(=O)(O)O)c1cc(C(F)(F)F)cc(C(F)(F)F)c1]

### CROSS-REFERENCE TO RELATED APPLICATIONS

The present application is a U.S. National Phase application under 35 U.S.C. 371 of International Application No. PCT/CN2023/071901, filed on Jan. 12, 2023, which claims the benefit of Chinese Patent Application No. CN 202210032508.4, filed on Jan. 12, 2022. The entire disclosure of the aforementioned Chinese Patent Application is incorporated herein by reference.

### TECHNICAL FIELD

The present disclosure belongs to the field of pharmaceutical preparations, and in particular relates to a pharmaceutical composition comprising a neurokinin-1 antagonist prodrug compound and a preparation method therefor.

### BACKGROUND

Tachykinins are peptide ligands for neurokinin receptors. Neurokinin receptors, such as NK1, NK2 and NK3, are involved in various biological processes. They can be found in the nervous and circulatory systems of mammals, as well as in surrounding tissues. Therefore, the regulation of such receptors has been investigated for potential treatment or prevention of various physiological disorders, conditions or diseases in mammals.

On the other hand, drug-induced hemolysis is caused by the destruction of a large number of red blood cells due to immune factors after drugs enter the human body, and clinical signs of hemolysis include anemia, jaundice and soy sauce-colored urine. Drug-induced hemolytic anemia can be divided into the following three types: (1) drug-induced immune hemolytic anemia, which results in an antibody-mediated hemolytic reaction; (2) hemolytic anemia caused by the action of drugs on red blood cells with an inherited enzyme deficiency (e.g., G6PD deficiency); (3) hemolytic anemia caused by a drug-induced hemolytic reaction to abnormal hemoglobin. The key to treating this disease is to discontinue the use of related drugs and control the occurrence of hemolysis so as to prevent complications.

WO 2020259675 provides a new NK1 antagonist prodrug compound (formula I) that is effective in the treatment of various physiological disorders, conditions or diseases with minimal side effects, and that has good pharmaceutical activity.

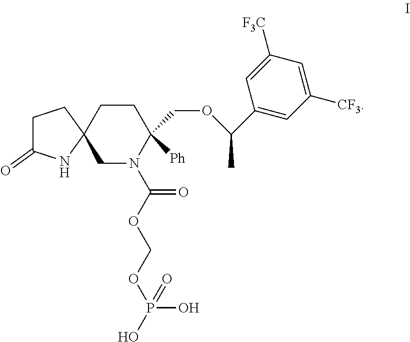 [SMILES: C[C@@H](OC[C@@]1(c2ccccc2)CC[C@]2(CCC(=O)N2)CN1C(=O)OCOP(=O)(O)O)c1cc(C(F)(F)F)cc(C(F)(F)F)c1]

### CONTENT OF THE PRESENT INVENTION

An objective of the present disclosure is to provide a pharmaceutical composition comprising a compound of formula I or a pharmaceutically acceptable salt thereof, which composition has excellent stability.

An aspect of the present disclosure provides a pharmaceutical composition comprising a compound of formula I or a pharmaceutically acceptable salt thereof and a stabilizer, wherein the stabilizer is an amino acid and/or a saccharide,

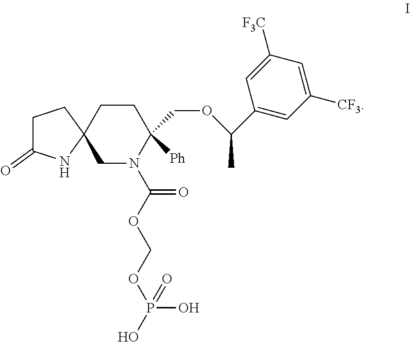 [SMILES: C[C@@H](OC[C@@]1(c2ccccc2)CC[C@]2(CCC(=O)N2)CN1C(=O)OCOP(=O)(O)O)c1cc(C(F)(F)F)cc(C(F)(F)F)c1]

In some embodiments, the amino acid is an amino acid that contains at least one carboxylic acid group [—C(═O)OH] and at least one primary amino group or secondary amino group [—NH₂ or —RNH, wherein —R is a group other than —H] but does not contain a secondary amide group [—C(═O)—NH—]. For example, the amino acid may be one or more of alanine, 4-aminobutyric acid, 3-aminopentanoic acid, 5-aminopentanoic acid, 6-aminohexanoic acid, 8-aminooctanoic acid, arginine, aspartic acid, asparagine, cysteine, glutamic acid, glutamine, glycine, histidine, isoleucine, leucine, lysine, methionine, methyllysine, ornithine, phenylalanine, proline, serine, threonine, tryptophan, tyrosine and valine.

In some embodiments, the amino acid is a basic amino acid, preferably one or more of arginine, lysine and histidine, and more preferably arginine.

In some embodiments, the saccharide is selected from one or more of sucrose, glucose, lactose, trehalose and maltose, preferably sucrose and/or glucose, and more preferably glucose.

In some embodiments, the stabilizer is selected from one or more of arginine, glucose and sucrose, more preferably arginine and/or glucose.

In some embodiments, the pharmaceutical composition further comprises a solvent. Available solvents include, but are not limited to, water, organic solvents, water/organic solvent cosolvent systems. The organic solvents include, but are not limited to, methanol, ethanol, n-propanol, isopropanol, n-butanol, isobutanol, tert-butanol, acetone, acetonitrile, dichloromethane, dimethyl sulfoxide, etc. Preferred solvents may be water, ethanol, isopropanol, tert-butanol, water/ethanol, water/isopropanol, water/tert-butanol, etc.

In some embodiments, the weight ratio of the compound of formula I or the pharmaceutically acceptable salt thereof to the stabilizer is 1:0.1-1:20, e.g., 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20 or any value therebetween, preferably 1:1-1:10, and more preferably 1:1-1:7.5.

In some embodiments, the compound of formula I or the pharmaceutically acceptable salt thereof has a mass volume percentage of 0.01%-30%, e.g., 0.01%, 0.05%, 0.1%, 0.2%, 0.5%, 1%, 2%, 3%, 4%, 5%, 6%, 7%, 8%, 9%, 10%, 12%, 14%, 16%, 18%, 20%, 22%, 24%, 26%, 28%, 30% or any value therebetween, preferably 0.01%-10%, and more preferably 0.1%-5%.

In some embodiments, the stabilizer has a mass volume percentage of 0.01%-30%, e.g., 0.01%, 0.05%, 0.1%, 0.2%, 0.5%, 1%, 2%, 3%, 4%, 5%, 6%, 7%, 8%, 9%, 10%, 12%, 14%, 16%, 18%, 20%, 22%, 24%, 26%, 28%, 30% or any value therebetween, preferably 0.01%-20%, and more preferably 0.1%-10%.

In some embodiments, the pharmaceutical composition further comprises a buffer. The buffer may be a phosphate buffer, a citrate buffer, an acetate buffer, a succinate buffer, a histidine salt buffer or other organic acid salt buffers.

In some embodiments, the buffer has a concentration of 5 mM-40 mM, preferably 10 mM-30 mM.

In some embodiments, the pharmaceutical composition may further comprise a pH regulator. The pH regulator includes, but is not limited to, sodium hydroxide, hydrochloric acid, etc.

In some embodiments, the pharmaceutical composition further comprises a chelating agent. The chelating agent is selected from ethylenediamine tetraacetic acid or a salt thereof.

In some embodiments, the weight ratio of the compound of formula I or the pharmaceutically acceptable salt thereof to the chelating agent is 1000:1-1:1, e.g., 1000:1, 900:1, 800:1, 700:1, 600:1, 500:1, 400:1, 300:1, 200:1, 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1 or any value therebetween, more preferably 1000:1-10:1. In some embodiments, the chelating agent has a mass volume percentage of 0.001%-5%, preferably 0.01%-2%.

In some embodiments, the pharmaceutical composition has a pH value of 3.0-8.0, e.g., 3.0, 3.5, 4.0, 4.5, 5.0, 5.5, 6.0, 6.5, 7.0, 7.5, 8.0 or any value therebetween, preferably 5.0-8.0.

In some embodiments, the pharmaceutical composition further comprises a 5-HT3 receptor antagonist selected from granisetron, ondansetron, ramosetron, tropisetron, palonosetron or dolasetron, preferably palonosetron.

In some embodiments, the weight ratio of the compound of formula I or the pharmaceutically acceptable salt thereof to the 5-HT3 receptor antagonist is 2000:1-10:1, e.g., 2000:1, 1900:1, 1800:1, 1700:1, 1600:1, 1500:1, 1400:1, 1300:1, 1200:1, 1100:1, 1000:1, 900:1, 800:1, 700:1, 600:1, 500:1, 400:1, 300:1, 200:1, 100:1, 90:1, 80:1, 70:1, 60:1, 50:1, 45:1, 40:1, 35:1, 30:1, 25:1, 20:1, 15:1, 10:1 or any value therebetween, preferably 1000:1-100:1. In some embodiments, the 5-HT3 receptor antagonist has a mass volume percentage of 0.0001%-1%, e.g., 0.0001%, 0.001%, 0.01%, 0.05%, 0.1%, 0.2%, 0.3%, 0.4%, 0.5%, 0.6%, 0.7%, 0.8%, 0.9%, 1% or any value therebetween, preferably 0.0001%-0.1%.

In some embodiments, the pharmaceutical composition comprises (based on mass volume percentage):
0.01%-10% of a compound of formula I or a pharmaceutically acceptable salt thereof;
0.01%-20% of a stabilizer selected from one or more of arginine, glucose and sucrose;
optionally 0.0001%-1% of palonosetron or a pharmaceutically acceptable salt thereof;
optionally a 5 mM-40 mM buffer;
optionally 0.01%-2% of a chelating agent; and
70%-99% of a solvent selected from water and/or tert-butanol.

The pharmaceutical composition may have a pH value of 3.0-8.0, preferably 5.0-8.0. The pharmaceutical composition may optionally further comprise a pH regulator, etc.

The present disclosure further provides a sterile powder obtained by freeze-drying or spray-drying the pharmaceutical composition of the present disclosure.

The present disclosure further provides a sterile powder, which is reconstituted to obtain the pharmaceutical composition of the present disclosure.

In some embodiments, the sterile powder of the present disclosure contains less than 5% of prototype drug impurities, and the content thereof may be less than 4.9%, 4.8%, 4.7%, 4.6%, 4.5%, 4.4%, 4.3%, 4.2%, 4.1%, 4.0%, 3.9%, 3.8%, 3.7%, 3.6%, 3.5%, 3.4%, 3.3%, 3.2%, 3.1%, 3.0%, 2.9%, 2.8%, 2.7%, 2.6%, 2.5%, 2.4%, 2.3%, 2.2%, 2.1%, 2.0% or less.

In some embodiments, the sterile powder of the present disclosure contains less than 5% of prototype drug impurities after being placed at 25° C. and 60% RH for 30 days, and the content thereof may be less than 4.9%, 4.8%, 4.7%, 4.6%, 4.5%, 4.4%, 4.3%, 4.2%, 4.1%, 4.0%, 3.9%, 3.8%, 3.7%, 3.6%, 3.5%, 3.4%, 3.3%, 3.2%, 3.1%, 3.0%, 2.9%, 2.8%, 2.7%, 2.6%, 2.5%, 2.4%, 2.3%, 2.2%, 2.1%, 2.0% or less.

The present disclosure further provides a reconstituted solution obtained by reconstituting the sterile powder of the present disclosure.

In some embodiments, the reconstituted solution is an injectable composition.

The reconstituted solution may have a pH value of 3.0-8.0, preferably 5.0-8.0. The reconstituted solution may use water for injection, physiological saline, a glucose solution, etc.

The present disclosure further provides a method for preparing the pharmaceutical composition of the present disclosure, wherein the method comprises the step of mixing a compound of formula I or a pharmaceutically acceptable salt thereof with a stabilizer.

In some embodiments, the method further comprises the step of drying the composition comprising the compound of formula I or the pharmaceutically acceptable salt thereof and the stabilizer. The drying method may be selected from freeze-drying or spray-drying.

In some embodiments, the stabilizer is formulated in the form of a solution. Preferably, the stabilizer solution has a pH<7.

The compound of formula I or the pharmaceutically acceptable salt thereof of the present disclosure may be a compound of formula I in any form, specific forms including, but not limited to, amorphous form, any crystal form, hydrate, solvate, etc.

The present disclosure further provides the use of the aforementioned pharmaceutical composition, sterile powder or reconstituted solution in the preparation of a drug for treating physiological disorders, conditions or diseases in a patient, wherein the physiological disorders, conditions or diseases are preferably respiratory diseases, cough, inflammatory diseases, skin disorders, ophthalmic disorders, depression, anxiety, phobia, bipolar disorder, alcohol dependence, neuroactive substance abuse, epilepsy, nociception, psychosis, schizophrenia, Alzheimer's disease, AIDS-related dementia, Towne's disease, stress-related disorders, obsessive/compulsive disorders, bulimia, anorexia nervosa, binge eating, mania, premenstrual syndrome, gastrointestinal dysfunction, atherosclerosis, fibrotic disorder, obesity, type II diabetes, headache, neuropathic pain, post-exercise pain, chronic pain syndrome, bladder disorders, urogenital disorders, vomiting or nausea.

The “mass volume percentage” (w/v) of the present disclosure refers to the mass (in g) of the component contained per 100 mL of a liquid system, i.e., g/100 mL.

The content (including percentage content) of various substances and the ratios of the various substances to each other of the present disclosure all have an allowable error of ±5%. For example, the expression “the content of the compound of formula I or the pharmaceutically acceptable salt thereof in the composition is 5 to 20 mg/mL” means that the content of the compound of formula I or the pharmaceutically acceptable salt thereof in the composition is 4.75 to 21 mg/mL, which falls within the scope of the present disclosure; the expression “the weight ratio of the compound of formula I or the pharmaceutically acceptable salt thereof to the stabilizer is selected from 1:1 to 1:100” means that the weight ratio of the compound of formula I or the pharmaceutically acceptable salt thereof to the stabilizer is selected from 1:0.95 to 1:105, which falls within the scope of the present disclosure.

The expressions “mixing . . . into . . . ” and “mixing . . . with . . . ” of the present disclosure mean that the adding sequence of components is not limited. For example, “mixing A into B” can mean adding A to B, and can also mean adding B to A, and “mixing A with B” can mean adding A to B for mixing, and can also mean adding B to A for mixing.

### DETAILED DESCRIPTION OF THE PREFERRED EMBODIMENT

### Example 1

According to the ratio (w/v) in the table below, 90% of the target volume of a solvent was taken, a prescription amount of a stabilizer was added to the solvent, the pH of the resulting solution was adjusted with hydrochloric acid to pH<7, to the solution was added the raw material of the compound of formula I, and the resulting mixture was stirred for dissolution at room temperature. The pH was adjusted with dilute hydrochloric acid or sodium hydroxide, filtration was performed with a 45 μm organic filtration membrane to obtain a clear solution, and the volume of the clear solution was made up to with the solvent. The above-mentioned solution was stored at 4° C. before subpackaging. After subpackaging according to the target volume, half of a stopper was pressed, and a freeze-drying procedure was run. After the completion of the procedure, nitrogen was filled, the remaining stopper was pressed, and the freeze-dried compounds were taken out of the box.

| | Content of | | | | | Other |
|---|---|---|---|---|---|---|
| | compound of | | Content of | Solvent | System | auxiliary |
| No. | formula I | Stabilizer | stabilizer | system | pH | materials |
| 1 | 2% | Sucrose | 5% | Water | 8 | |
| 2 | 2% | Glucose | 5% | Water | 8 | |
| 3 | 2% | Lactose | 2% | Water | 8 | |
| 4 | 2% | Arginine | 5% | Water | 8 | |
| 5 | 2% | Histidine | 2% | Water | 8 | |
| 6 | 2% | Proline | 5% | Water | 8 | |
| 7 | 2% | SBECD | 5% | Water | 8 | |
| 8 | 2% | Hydroxyethyl | 2% | Water | 8 | |
| | | starch | | | | |
| 9 | 2% | PEG 4000 | 2% | Water | 8 | |
| 10 | 2% | PVP K12 | 2% | Water | 8 | |
| 11 | 2% | Dextran 40k | 2% | Water | 8 | |
| 12 | 4.36% | Trehalose | 5% | 50% TBA | 5 | |
| 13 | 4.36% | Mannitol | 5% | 50% TBA | 5 | |
| 14 | 4.35% | NADA | 8% | 50% TBA | 5 | |
| 15 | 3.00% | Maltose | 5% | 50% TBA | 5 | |
| 16 | 3.0% | Arginine | 5% | 50% TBA | 5 | 20 mM citrate |
| | | | | | | buffer |
| 17 | 2.1% | Arginine | 5% | Water | 8 | 0.05% |
| | | | | | | EDTA-2NaCa |
| 18 | 3.00% | Arginine | 5% | 50% TBA | 3 | |
| 19 | 3.00% | Arginine | 5% | 50% TBA | 4 | |
| 20 | 3.00% | Arginine | 5% | 50% TBA | 5 | |
| 21 | 3.00% | Arginine | 5% | 50% TBA | 6 | |
| 22 | 3.00% | Arginine | 5% | 50% TBA | 7 | |
| 23 | 3.00% | Arginine | 5% | 50% TBA | 8 | |
| 24 | 3.00% | Sucrose | 5% | 50% TBA | 5 | |
| 25 | 3.00% | Sucrose | 5% | 50% TBA | 8 | |
| 26 | 3.00% | Glucose | 3% | 50% TBA | 5 | |
| 27 | 3.00% | Glucose | 3% | 50% TBA | 8 | |

50% TBA: 50% aqueous tert-butanol solution;
SBECD: sulfobutylether-β-cyclodextrin;
PVP: polyvinylpyrrolidone;
NADA: N-acetyl-D-alanine

### Example 2

The preparation samples prepared in example 1 were placed under accelerated conditions (25° C., 60% RH), and were detected for the active substance and impurity content. Detection method: detection was performed by a high performance liquid chromatography system, wherein the chromatographic column was ACE Excel 3 SuperC18, 4.6×150 mm, 3 μm, the mobile phase A was 0.01 mol/L potassium dihydrogen phosphate solution-pH 7.0, the mobile phase B was acetonitrile, and the detection wavelength was 210 nm.

The detection results are as shown in the table below. The prototype drug impurity is rolapitant formed by the degradation of the compound of formula I.

| | | | | | | Prototype |
|---|---|---|---|---|---|---|
| | | | | | | drug |
| | | | | | Purity | impurity |
| No. | Stabilizer | Stability condition | | | (%) | (%) |
| 1 | Sucrose | 0 | 0 | d | 99.33 | 0.46\grave{ } |
| | | 25° C./60% RH | 14 | d | 98.82 | 0.94 |
| | | | 30 | d | 98.54 | 1.24 |
| | | | 60 | d | 98.19 | 1.63 |
| 2 | Glucose | 0 | 0 | d | 99.42 | 0.36 |
| | | 25° C./60% RH | 14 | d | 99.38 | 0.48 |
| | | | 30 | d | 99.15 | 0.72 |
| | | | 60 | d | 98.47 | 1.37 |
| | | | 90 | d | 97.73 | 1.89 |
| 3 | Lactose | 0 | 0 | d | 98.4 | 1.04 |
| | | 25° C./60% RH | 14 | d | 95.38 | 3.16 |
| 4 | Arginine | 0 | 0 | d | 99.43 | 0.37 |
| | | 25° C./60% RH | 14 | d | 99.25 | 0.57 |
| | | | 30 | d | 99.14 | 0.69 |
| | | | 60 | d | 98.92 | 0.89 |
| | | | 90 | d | 98.83 | 0.98 |
| 5 | Histidine | 0 | 0 | d | 98.37 | 1.12 |
| | | 25° C./60% RH | 14 | d | 94.58 | 3.89 |
| | | | 90 | d | 88.43 | 9.01 |
| 6 | Proline | 0 | 0 | d | 99.39 | 0.43 |
| | | 25° C./60% RH | 14 | d | 96.53 | 2.79 |
| | | | 90 | d | 50.71 | 40.80 |
| 7 | SBECD | 0 | 0 | d | 93.28 | 3.6 |
| | | 25° C./60% RH | 14 | d | 81.15 | 10.92 |
| | | | 90 | d | 63.95 | 22.77 |
| 8 | Hydroxyethyl | 0 | 0 | d | 91.90 | 3.83 |
| | starch | 25° C./60% RH | 14 | d | 82.069 | 9.55 |
| 9 | PEG 4000 | 0 | 0 | d | 90.44 | 4.74 |
| | | 25° C./60% RH | 14 | d | 78.772 | 10.881 |
| 10 | PVP K12 | 0 | 0 | d | 88.20 | 5.81 |
| | | 25° C./60% RH | 14 | d | 86.121 | / |
| 11 | Dextran | 0 | 0 | d | 92.79 | 3.59 |
| | 40k | 25° C./60% RH | 14 | d | 82.914 | 9.168 |
| 12 | Trehalose | 0 | 0 | d | 99.087 | 0.504 |
| | | 25° C./60% RH | 7 | d | 98.36 | 1.06 |
| | | | 14 | d | 97.63 | 1.58 |
| 13 | Mannitol | 0 | 0 | d | 98.949 | 0.494 |
| | | 25° C./60% RH | 7 | d | 98.09 | 1.03 |
| | | | 14 | d | 97.19 | 1.56 |
| 14 | NADA | 0 | 0 | d | 98.87 | 0.39 |
| | | 25° C./60% RH | 7 | d | 97.85 | 1.01 |
| | | | 14 | d | 97.15 | 1.45 |
| | | | 30 | d | 95.65 | 2.36 |
| 15 | Maltose | 0 | 0 | d | 99.24 | 0.41 |
| | | 25° C./60% RH | 7 | d | 98.48 | 0.91 |
| | | | 14 | d | 97.80 | 1.42 |
| | | | 30 | d | 96.95 | 2.03 |
| 16 | Arginine | 0 | 0 | d | 99.19 | 0.43 |
| | | 25° C./60% RH | 7 | d | 99.13 | 0.51 |
| | | | 14 | d | 98.91 | 0.63 |
| | | | 30 | d | 98.72 | 0.74 |
| | | | 60 | d | 98.34 | 0.97 |
| | | | 90 | d | 98.12 | 1.21 |
| 17 | Arginine | 0 | 0 | d | 99.53 | 0.38 |
| | | 25° C./60% RH | 7 | d | 98.88 | 0.90 |
| | | | 14 | d | 98.81 | 0.95 |
| | | | 30 | d | 99.10 | 0.78 |
| | | | 90 | d | 98.72 | 1.00 |
| 18 | Arginine | 0 | 0 | d | 99.21 | 0.40 |
| | | 25° C./60% RH | 07 | d | 98.86 | 0.64 |
| | | | 14 | d | 98.49 | 0.76 |
| | | | 30 | d | 97.95 | 1.24 |
| | | | 60 | d | 97.33 | 1.82 |
| | | | 90 | d | 96.51 | 2.74 |
| 19 | Arginine | 0 | 0 | d | 99.24 | 0.41 |
| | | 25° C./60% RH | 07 | d | 98.92 | 0.63 |
| | | | 14 | d | 98.61 | 0.80 |
| | | | 30 | d | 98.31 | 1.01 |
| | | | 60 | d | 97.72 | 1.44 |
| | | | 90 | d | 97.10 | 1.99 |
| 20 | Arginine | 0 | 0 | d | 99.24 | 0.40 |
| | | 25° C./60% RH | 07 | d | 98.94 | 0.60 |
| | | | 14 | d | 98.71 | 0.75 |
| | | | 30 | d | 98.36 | 0.96 |
| | | | 60 | d | 97.75 | 1.37 |
| | | | 90 | d | 97.25 | 1.88 |
| 21 | Arginine | 0 | 0 | d | 99.25 | 0.39 |
| | | 25° C./60% RH | 07 | d | 98.96 | 0.58 |
| | | | 14 | d | 98.78 | 0.69 |
| | | | 30 | d | 98.31 | 1.02 |
| | | | 60 | d | 97.86 | 1.36 |
| | | | 90 | d | 97.84 | 1.4 |
| 22 | Arginine | 0 | 0 | d | 99.27 | 0.37 |
| | | 25° C./60% RH | 07 | d | 98.92 | 0.61 |
| | | | 14 | d | 98.75 | 0.88 |
| | | | 30 | d | 98.34 | 1.06 |
| | | | 60 | d | 97.88 | 1.40 |
| | | | 90 | d | 97.56 | 1.76 |
| 23 | Arginine | 0 | 0 | d | 99.20 | 0.43 |
| | | 25° C./60% RH | 07 | d | 98.69 | 0.80 |
| | | | 14 | d | 98.60 | 0.89 |
| | | | 30 | d | 98.32 | 1.11 |
| | | | 60 | d | 97.58 | 1.64 |
| | | | 90 | d | 97.24 | 2.02 |
| 24 | Sucrose | 0 | 0 | d | 99.25 | 0.39 |
| | | 25° C./60% RH | 07 | d | 98.53 | 0.94 |
| | | | 14 | d | 98.08 | 1.34 |
| | | | 30 | d | 97.22 | 1.98 |
| 25 | Sucrose | 0 | 0 | d | 98.74 | 0.69 |
| | | 25° C./60% RH | 07 | d | 97.58 | 1.53 |
| | | | 14 | d | 96.61 | 2.18 |
| | | | 30 | d | 95.91 | 2.76 |
| 26 | Glucose | 0 | 0 | d | 99.24 | 0.41 |
| | | 25° C./60% RH | 07 | d | 98.97 | 0.62 |
| | | | 14 | d | 98.63 | 0.90 |
| | | | 30 | d | 98.03 | 1.23 |
| | | | 60 | d | 97.34 | 1.85 |
| | | | 90 | d | 95.82 | 3.37 |
| 27 | Glucose | 0 | 0 | d | 98.95 | 0.58 |
| | | 25° C./60% RH | 07 | d | 97.95 | 1.26 |
| | | | 14 | d | 97.93 | 1.31 |
| | | | 30 | d | 97.35 | 1.70 |
| | | | 60 | d | 97.40 | 1.87 |
| | | | 90 | d | 96.17 | 2.71 |

After being placed under accelerated conditions, the freeze-dried preparations in which the stabilizers are sucrose, glucose and arginine have little change in purity, and are low in the prototype drug impurity from degradation, indicating good stability.

### Example 3

A compound freeze-dried preparation comprising the compound of formula I and palonosetron hydrochloride was prepared using the method of example 1 according to the ratio (w/v) in the table below.

| | Content of | Content of | | | | | |
|---|---|---|---|---|---|---|---|
| | compound of | palonosetron | | Solvent | System | | Chelating |
| No. | formula I | hydrochloride | Stabilizer | system | pH | Buffer | agent |
| 1 | 3.11% | 0.004011% | 5% | Water | 6.5 | 20 mM | 0.5% |
| | | | Arginine | | | Citrate | EDTA- |
| | | | | | | | 2NaCa |

### Example 4

The preparation sample prepared in example 3 was placed under accelerated conditions (25° C., 60% RH), and was detected for the active substance and impurity content. The results are as shown in the table below.

| | | | | Total impurity | Prototype drug |
|---|---|---|---|---|---|
| No. | Stability condition | | | content (%) | impurity (%) |
| 1 | 0 | 0 | d | 1.18 | 0.37 |
| | 25° C./60% RH | 7 | d | 1.32 | 0.53 |
| | | 1 | M | 1.42 | 0.82 |
| | | 2 | M | 1.48 | 1.07 |

What is claimed is:

1. A pharmaceutical composition, comprising a compound of formula I or a pharmaceutically acceptable salt thereof and a stabilizer, wherein the stabilizer is selected from an amino acid and a saccharide,

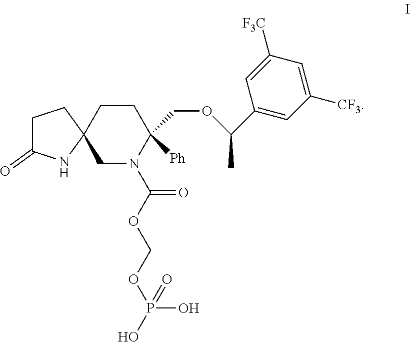 [SMILES: C[C@@H](OC[C@@]1(c2ccccc2)CC[C@]2(CCC(=O)N2)CN1C(=O)OCOP(=O)(O)O)c1cc(C(F)(F)F)cc(C(F)(F)F)c1]

2. The pharmaceutical composition according to claim 1, wherein the amino acid is an amino acid that contains at least one carboxylic acid group and at least one primary amino group or secondary amino group but does not contain a secondary amide group, preferably alanine, 4-aminobutyric acid, 3-aminopentanoic acid, 5-aminopentanoic acid, 6-aminohexanoic acid, 8-aminooctanoic acid, arginine, aspartic acid, asparagine, cysteine, glutamic acid, glutamine, glycine, histidine, isoleucine, leucine, lysine, methionine, methyllysine, ornithine, phenylalanine, proline, serine, threonine, tryptophan, tyrosine and valine-;
or, the amino acid is a basic amino acid, preferably arginine, lysine and histidine, and more preferably arginine; or
or, the saccharide is selected from one or more of sucrose, glucose, lactose, trehalose and maltose, preferably sucrose or glucose, and more preferably glucose; or
or, the stabilizer is selected from one or more of arginine, glucose and sucrose.

3-5. (canceled)

6. The pharmaceutical composition according to claim 1, wherein the pharmaceutical composition further comprises a solvent, preferably the solvent is selected from water, an organic solvent or a water/organic solvent cosolvent system, and more preferably the solvent is selected from water, ethanol, isopropanol, tert-butanol, water/ethanol, water/isopropanol or water/tert-butanol.

7. The pharmaceutical composition according to claim 1, wherein the weight ratio of the compound of formula I or the pharmaceutically acceptable salt thereof to the stabilizer is 1:0.1-1:20, preferably 1:1-1:10, and more preferably 1:1-1:7.5.

8. The pharmaceutical composition according to claim 6, wherein the pharmaceutical composition meets one or more of the following conditions:
(1) the compound of formula I or the pharmaceutically acceptable salt thereof has a mass volume percentage of 0.01%-30%, preferably 0.01%-10%, and more preferably 0.1%-5%;
(2) the stabilizer has a mass volume percentage of 0.01%-30%, preferably 0.01%-20%, and more preferably 0.1%-10%.

9. (canceled)

10. The pharmaceutical composition according to claim 1, wherein the pharmaceutical composition meets one or more of the following conditions:
(1) the pharmaceutical composition further comprises a buffer, the buffer is preferably a phosphate buffer, a citrate buffer, an acetate buffer, a succinate buffer, a histidine salt buffer or other organic acid salt buffers, and preferably the buffer has a concentration of 5 mM-40 mM, more preferably 10 mM-30 mM;
(2) the pharmaceutical composition further comprises a pH regulator;
(3) the pharmaceutical composition further comprises a chelating agent, preferably the chelating agent is ethylenediamine tetraacetic acid or a salt thereof, preferably the chelating agent has a mass volume percentage of 0.001%-5%, more preferably 0.01%-2%, and preferably the weight ratio of the compound of formula I or the pharmaceutically acceptable salt thereof to the chelating agent is 1000:1-1:1, more preferably 1000:1-10:1;
(3) the pharmaceutical composition has a pH value of 3.0-8.0, preferably 5.0-8.0;
(4) the pharmaceutical composition further comprises a 5-HT3 receptor antagonist, preferably the 5-HT3 receptor antagonist is selected from granisetron, ondansetron, ramosetron, tropisetron, palonosetron or dolasetron, more preferably palonosetron, preferably the 5-HT3 receptor antagonist has a mass volume percentage of 0.0001%-1%, more preferably 0.0001%-0.1%, and preferably the weight ratio of the compound of formula I or the pharmaceutically acceptable salt thereof to the 5-HT3 receptor antagonist is 2000:1-10:1, more preferably 1000:1-100:1.

11-14. (canceled)

15. The pharmaceutical composition according to claim 1, comprising:
a compound of formula I or a pharmaceutically acceptable salt thereof;
a stabilizer selected from one or more of arginine, glucose and sucrose;
optionally palonosetron or a pharmaceutically acceptable salt thereof;
optionally a buffer;
optionally a chelating agent;
or comprising:
0.01%-10% of a compound of formula I or a pharmaceutically acceptable salt thereof;
0.01%-20% of a stabilizer selected from one or more of arginine, glucose and sucrose;
optionally 0.0001%-1% of palonosetron or a pharmaceutically acceptable salt thereof;
optionally a 5 mM-40 mM buffer;
optionally 0.01%-2% of a chelating agent;
70%-99% of a solvent selected from water or tert-butanol;
the percentages are based on mass volume percentages.

16. (canceled)

17. A sterile powder obtained by freeze-drying or spray-drying the pharmaceutical composition according to claim 1.

18. (canceled)

19. The sterile powder according to claim 17, wherein the sterile powder contains less than 5% of prototype drug impurities, preferably less than 4%, more preferably less than 3%, and most preferably less than 2%.

20. The sterile powder according to claim 17, wherein the sterile powder contains less than 5% of prototype drug impurities after being placed at 25° C. and 60% RH for 30 days.

21. A reconstituted solution obtained by reconstituting the sterile powder according to claim 17.

22. (canceled)

23. A method for treating physiological disorders, conditions or diseases in a subject in need thereof, comprising administering and effective amount of the pharmaceutical composition according to claim 1 to the subject, wherein the physiological disorders, conditions or diseases are preferably respiratory diseases, cough, inflammatory diseases, skin disorders, ophthalmic disorders, depression, anxiety, phobia, bipolar disorder, alcohol dependence, neuroactive substance abuse, epilepsy, nociception, psychosis, schizophrenia, Alzheimer's disease, AIDS-related dementia, Towne's disease, stress-related disorders, obsessive/compulsive disorders, bulimia, anorexia nervosa, binge eating, mania, premenstrual syndrome, gastrointestinal dysfunction, atherosclerosis, fibrotic disorder, obesity, type II diabetes, headache, neuropathic pain, post-exercise pain, chronic pain syndrome, bladder disorders, urogenital disorders, vomiting or nausea.
